(19) United States
(12) Patent Application Publication
(10) Pub. No.: US 2014/0283837 A1
(43) Pub. Date: Sep. 25, 2014

(54) High Concentration Nasal Oxygen Mask

(71) Applicant: Carmelo Thomas Turrisi, Jackson, NJ (US)

(72) Inventor: Carmelo Thomas Turrisi, Jackson, NJ (US)

(21) Appl. No.: 13/847,002

(22) Filed: Mar. 19, 2013

(51) Int. Cl. A61M 16/06; A61M 16/00; A61M 16/10; A61M 16/20

(52) U.S. Cl. CPC A61M 16/0616; A61M 16/208; A61M 16/0078; A61M 16/1005
USPC 128/205.13; 128/205.24

(57) ABSTRACT

A high concentration nasal oxygen mask for delivering high concentrations of oxygen to a patient via the nasal passages of the patient. The high concentration nasal oxygen mask includes a mask portion for enclosing the nose of the patient, an oxygen reserve bag for receiving a supply of oxygen from an external source, a one-way valve for allowing the passage of oxygen in only one direction from the reserve bag to the mask, and a one-way flap to allow the patient's expired gases to flow out of the mask without allowing atmospheric gases back into the mask.

### PRIORITY

This invention claims priority to Provisional Application 61/685,850 filed on Mar. 26, 2012.

### FIELD OF THE INVENTION

This invention relates to oxygen masks used on patients who are undergoing medical procedures where oxygen supplementation via the oral route is not possible.

### BACKGROUND OF THE INVENTION

Oxygen masks are commonly used to provide pure oxygen gas to patients undergoing medical procedures. Most of these masks are designed to cover the nasal and oral openings forming tight fit there around. Further, an oxygen supply line and reserve bag is used to provide a constant flow of oxygen gas. One of the disadvantages of the prior art configurations is that a patient's expired gases can only escape the mask if exhaust holes are provided through the mask. However, if exhaust holes are provided through the mask, then atmospheric gases will also enter the mask and become mixed with the provided oxygen and dilute the oxygen in the mask, thereby reducing the effectiveness of the oxygen gas and mask.

Another disadvantage of these prior art masks is that when a patient requires a procedure that includes the use of other hoses within the oral opening, or any other obstruction to the oral opening, a standard prior art mask cannot be used. Some of these procedures include transesophogeal echocardiograms, endoscopic gastroesophogeal duodenoscopies, and ERCPs.

Therefore, it became necessary to develop a new type of mask that could fit around a patient's nasal openings only, and provide a mechanism to exhaust the patient's expired gases without allowing external atmospheric gases to enter therein, thereby maintaining a high concentration of oxygen gas with the mask at all times.

### SUMMARY OF THE INVENTION

This invention essentially consists of a high concentration nasal oxygen mask including a mask portion for enclosing the nose of the patient, an oxygen reserve bag for receiving a supply of oxygen from an external source, a one-way valve for allowing the passage of oxygen in only one direction from the reserve bag to the mask, and a one-way flap to allow the patient's expired gases to flow out of the mask without allowing atmospheric gases back into the mask.

The mask can further include small airway apertures on one side of the mask to allow excess gasses to escape the mask with only a minimal amount of atmospheric gases entering back in.

One important advantage of this new inventive mask over prior oxygen masks is that a patient's expired gases can escape the mask without atmospheric gases entering the mask and diluting the oxygen content within the mask.

Another important advantage of this new inventive is that it can be used when a patient requires a procedure that includes the use of other hoses within the oral opening, or any other obstruction to the oral opening.

The novel features of the present invention are set forth in the claims herein. The functionality, modes of operation, purposes, and advantages of this invention can be further understood with reference to the following drawings and description of the preferred embodiment.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be made to the accompanying drawings and descriptive matter which contains illustrated preferred embodiments of the invention.

### BRIEF DESCRIPTION OF THE DRAWINGS

FIG. 1 is a side view of a patient wearing the inventive mask.

FIG. 2 is a front view of the inventive mask.

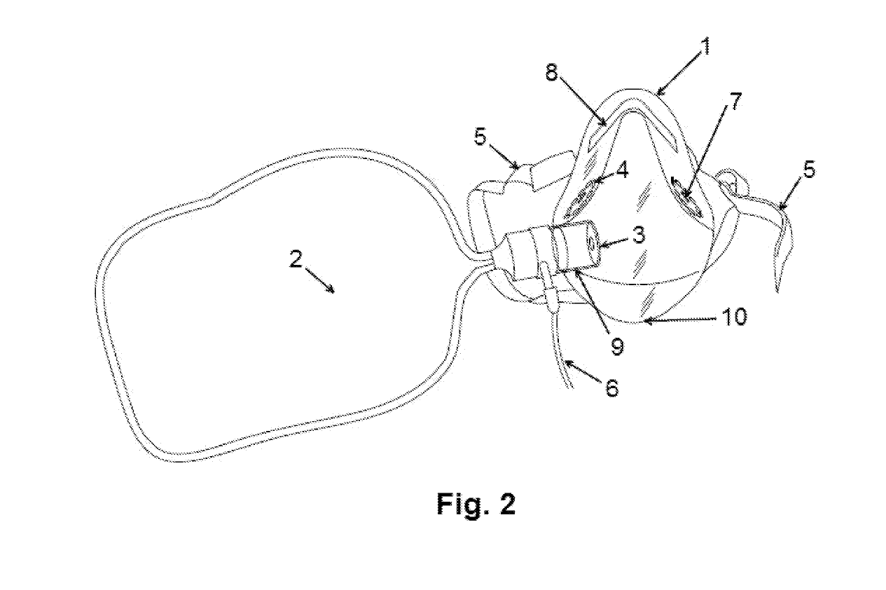

FIG. 3 is a perspective view of the inventive mask.

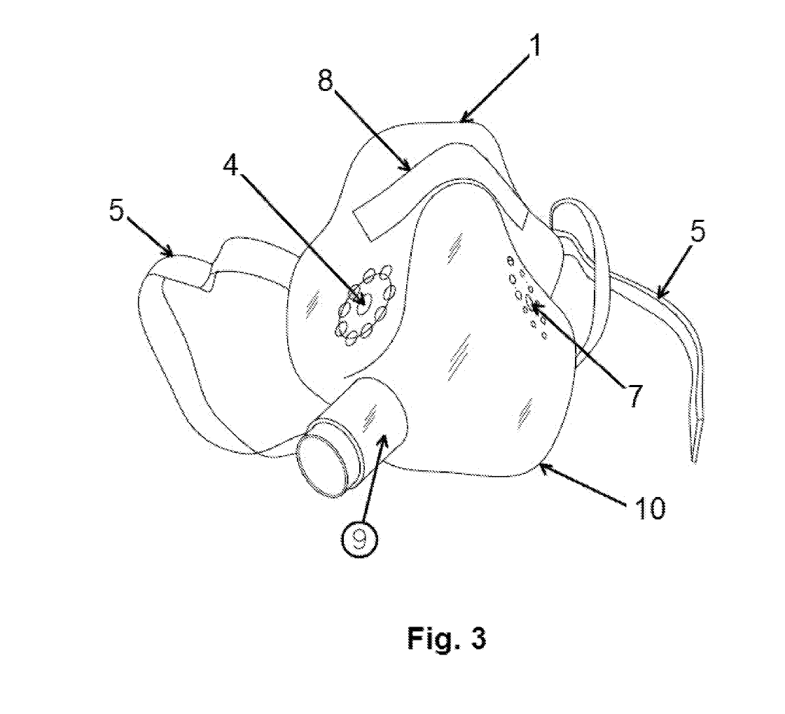

### DETAILED DESCRIPTION OF THE PREFERRED EMBODIMENT

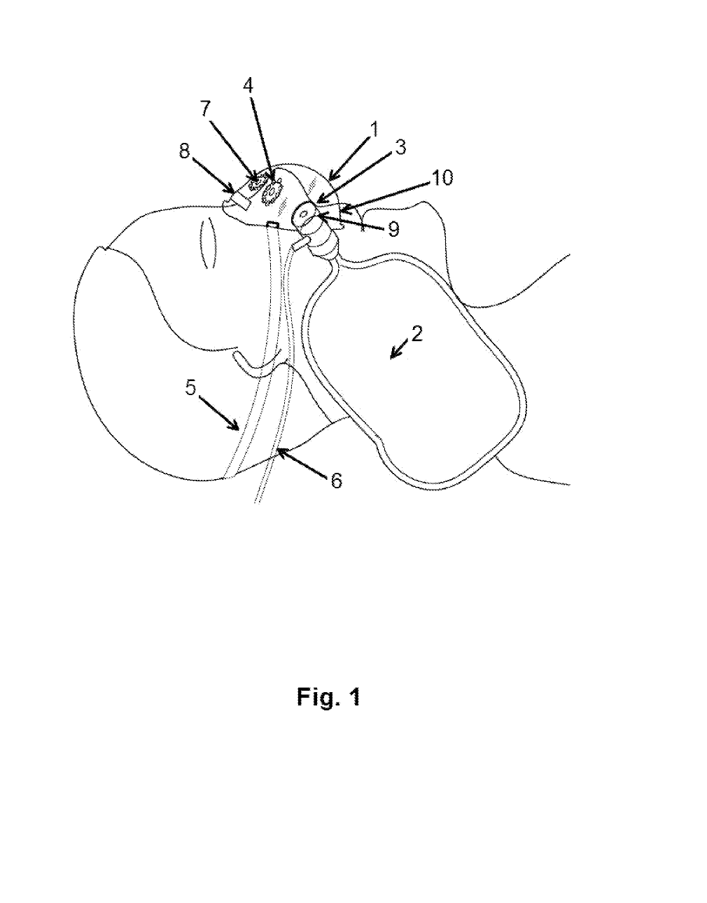

As per FIG. 1, the inventive mask includes a mask portion (1) for enclosing the nose of a patient; an oxygen reserve bag (2) attached to a right hand side of the mask portion and for receiving a supply of oxygen from an external source and providing the oxygen to the mask portion; a one-way valve (3) for allowing the passage of oxygen in only one direction from the reserve bag to the mask portion; a one-way flap (4) to allow the patient's expired gases to flow out of the mask without allowing atmospheric gases back into the mask; an adjustable elastic strap (5) attached on opposite sides of the mask portion and used to hold the mask firmly against a patients face covering their nasal passages; a supply tube (6) connected between the oxygen gas supply and the oxygen reserve bag and for delivering the oxygen therebetween; small apertures (7) located through an opposite side of the mask from where the oxygen reserve bag is attached and used to allow excess expired gases to escape the mask with only a minimal amount of atmospheric gases allowed back therein; a pliable metal strap (8) attached to a center section of the mask portion and used to be bent around the bridge of a patient's nose for helping secure a snug fit there around; a sleeve (9) placed between the oxygen reserve bag and the mask portion and adapted to house the one-way valve such that when the patient breathes inward the oxygen from the oxygen reserve bag flows through the sleeve (9), pushes the one-way valve open, and continues into the mask portion, and such that when the patient breathes outward the one-way valve (9) is pushed closed and does not allow the expired gases to flow into the oxygen reserve bag; and an inferior border (10) that forms a section of the mask portion that is adapted to fit snugly upon and above the upper lip of a patient's mouth.

As identified previously, procedures that include transesophogeal echocardiograms, endoscopic gastroesophogeal duodenoscopies, and ERCPs, the patient is positioned left lateral decubitus, prone, or supine with the anesthesiologist positioned at the patient's right side, therefore the oxygen source is usually positioned to the right side of the oxygen mask.

The instant mask portion can be formed from soft, pliable material to ensure its non-traumatic nature and allows it to be adapted to be used in conjunction with a nasal end-tidal carbon dioxide (EtCO2) monitor, which is required for patients under “Total Intravenous Sedation” or “General Anesthesia”.

The instant mask portion can also be formed from a sturdier material, such as hard plastics, to be fitted over the nose with a soft plastic lining placed where it comes in contact with the patient's face. This can also allow for a flexible attachment of the one-way valve between the oxygen reserve bag and the mask portion.

As to a further discussion of the manner of usage and operation of the present invention, the same should be apparent from the above description. Accordingly, no further discussion relating to the manner of usage and operation will be provided.

With respect to the above description, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed is:

1. A high concentration oxygen mask comprising a mask portion adapted to enclose the nasal passages of a medical patient; a sleeve including a one-way valve therein, said sleeve attached to one side of said mask portion and adapted such that when said medical patient breathes inward oxygen flows through said sleeve, pushes the one-way valve open, and continues into the mask portion, and adapted such that when said medical patient breathes outward said one-way valve is pushed closed and does not allow expired gases to flow through said sleeve; and a one-way flap located on said one side of said mask portion and is adapted to open and close allowing expired gases of said medical patient to flow out from said mask portion without allowing atmospheric gases back into said mask portion.

2. The high concentration oxygen mask of claim 1, further comprising at least one small aperture located through an opposite side from said one side of said mask portion and are adapted to allow excess expired gases from said medical patient to escape the mask with only a minimal amount of atmospheric gases allowed back therethrough.

3. The high concentration oxygen mask of claim 2, further comprising an oxygen reserve bag attached to a distal end of said sleeve opposite said mask portion, said oxygen reserve bag adapted to receive a supply of oxygen from an external source and aid in supplying a measured flow of oxygen through said sleeve; and a supply tube attached to a portion of said sleeve in between said distal end and an end attached to said mask portion, said supply tube adapted to transport a supply of oxygen from an external source to said sleeve and said oxygen reserve bag.

4. The high concentration oxygen mask of claim 3, further comprising an adjustable elastic strap having opposite ends attached to opposite sides of said mask portion and adapted to hold said mask portion firmly against said medical patient's face covering their nasal passages.

5. The high concentration oxygen mask of claim 4, further comprising a pliable metal strap attached to a center section of said mask portion and adapted to be bent around the bridge of said medical patient's nose for helping secure a snug fit there around.

6. The high concentration oxygen mask of claim 5, further comprising an inferior border that forms a section of said mask portion that is adapted to fit snugly upon and above the upper lip of said medical patient's mouth.

7. The high concentration oxygen mask of claim 2, wherein said at least one small aperture is a plurality of apertures.

8. The high concentration oxygen mask of claim 1, wherein said mask portion is formed from a soft, pliable plastic material.

9. The high concentration oxygen mask of claim 1, wherein said mask portion is formed from a rigid plastic material and adapted to incorporate a soft plastic lining in the area of said mask portion that is adapted to make contact with said medical patient's face.

10. The high concentration oxygen mask of claim 1, wherein said one side of said mask portion is the side adapted to cover the right side of said medical patient's nasal passages and face, such that said sleeve is adapted to be positioned next to an anesthesiologist that is usual positioned on the right hand side of medical patients during medical procedures.

11. The high concentration oxygen mask of claim 1, wherein said sleeve is formed in a cylindrical shape; and said one-way valve is formed in a disc-shape, and sized to fit snugly within the interior of said sleeve.

12. The high concentration oxygen mask of claim 1, wherein said one-way flap is formed in a disc-shape.
